(19) United States
(12) Patent Application Publication
(10) Pub. No.: US 2015/0064705 A1
(43) Pub. Date: Mar. 5, 2015

(54) DYE SETS FOR SURFACE ENHANCED RESONANT RAMAN SPECTROSCOPY

(71) Applicant: RENISHAW DIAGNOSTICS LIMITED, Glasgow, Strathclyde (GB)

(72) Inventors: Alastair Ricketts, Glasgow (GB); Karen Fitchett, West Way (GB); Julie Green, Lanark (GB); Graeme McNay, Glasgow (GB); Brian Smith, Dursley (GB); Thomas Thurston, Shirehampton (GB); Ian Bell, Cam (GB); Andrew Woolfrey, Wotton-under-Edge (GB)

(73) Assignee: Renishaw Diagnostics Limited, Glasgow, Strathclyde (GB)

(21) Appl. No.: 14/389,915

(22) PCT Filed: Apr. 2, 2013
(86) PCT No.: PCT/GB2013/050861

§ 371 Date: Oct. 1, 2014

(30) Foreign Application Priority Data
Apr. 5, 2012 (EP) 12163369.7
May 4, 2012 (GB) 1207821.8

(51) Int. Cl. C12Q 1/68; G01N 21/65; G01N 21/25

(52) U.S. Cl. CPC C12Q 1/6809; C12Q 1/6851; G01N 21/25; G01N 21/658
USPC 435/6.12; 435/287.2; 356/301; 702/19

(57) ABSTRACT

A kit for use in a multiplex assay, the kit including a dye set consisting essentially of a plurality of dyes and an association of each dye to a reference concentration. The dye set has been selected such that, using surface enhanced resonant Raman spectroscopy, each dye of the set is identifiable at better than 90% sensitivity and 90% specificity in the presence of any other dye of the set throughout a range of concentrations of each of the two dyes from 0.6 to 1.5 of the respective dye's reference concentration. In one embodiment, the dye set consists of 10 of the following dyes: JOE, Rhodamine Green, ATTO520, BODIPY FL, BODIPY TMR-X, FAM, HEX, Cy3, Cy3.5, TAMRA and TYE563. The invention also concerns a multiplex assay including the set of dyes, apparatus for carrying out the multiplex assay and a method of selecting the set of dyes.

### FIELD OF THE INVENTION

The present invention relates to dye sets for use in a multiplex assay, wherein the dyes are identified using surface enhanced resonant Raman spectroscopy (SERRS), and methods of selecting such dye sets. The present invention also relates to a multiplex assay and apparatus for carrying out the multiplex assay.

### BACKGROUND

Multiplexing within a laboratory environment is defined as the detection of multiple analytes simultaneously in a single interrogation. In general, the advantages are that many more data points are generated and a higher degree of information can be gathered. This can lead to a large time reduction which can be attained by searching for multiple targets in a single test rather than carrying out multiple, serial assays. High throughput multiplex assays mean that large scale testing is easily facilitated. In addition, the physical volume of sample required for a full panel of tests is significantly reduced this can be important in cases where the sample can be difficult to obtain and/or is only available in small volumes. A knock-on effect from this is that the amount of lab consumables and human resource is also significantly reduced, meaning a reduction in running costs and waste.

Many techniques can be used to detect multiple analytes. One of the most common techniques is fluorescence, where the analyte of interest is fluorescent or the analyte of interest is tagged with a fluorescent dye. There are several drawbacks to using fluorescence as a detection technique. The main problem is the broad nature of fluorescence emission spectra which lack uniquely identifiable features. This limits the number of analytes which can be simultaneously detected in a mixture due to the large spectral emission overlap that occurs between fluorophores.

Another molecular technique that gives molecularly characteristic spectra is Raman spectroscopy. Rather than the broad peaks observed with fluorescence, Raman produces narrow, well defined peaks which are molecularly specific and can be used to identify a molecule in situ in a mixture. These spectra are information rich and this specificity allows the opportunity for higher order multiplexing, however Raman scattering when used in its basic form lacks the sensitivity required for many real world multiplexed applications.

There are methods that can be used to enhance the signal. Surface enhanced Raman scattering (SERS), makes use of the plasmonic properties of metals to achieve enhancements in the Raman signal of up to 10⁵-10⁶. This allows multiplexing to occur at detection levels which may be of use in certain fields where direct identification of an analyte is required.

Surface enhanced resonance Raman scattering (SERRS) is another method of enhancing Raman signals, and uses the principles of SERS together with an analyte that contains a chromophore, where the chromophore has an electronic transition in the region of the laser wavelength being used to excite the sample. SERRS detection levels can surpass that seen for fluorescence (Faulds et al. Analyst, 129, 567-568 (2004)) by up to three orders of magnitude and combined with the selectivity of the technique make this an excellent technique for high order multiplexing. Each chromophore has a SERRS spectrum which is unique allowing it to be identified in situ.

Faulds et al. (Angew. Chem. Int. Ed., 46(11), 1829-1831 (2007)) have carried out a 5-plex of labelled oligonucleotide sequences where they identified 5 different dye labelled oligonucleotides in a mixture by careful choice of the dye and by using two excitation wavelengths. The sequences used corresponded to a range of different targets. FAM, Cy 5.5 and BODIPY TR-X were used to label a universal reverse primer, Rhodamine 6G (R6G) was used to label a probe for HPV, and ROX to label a probe to the VT2 gene of E. coli 157. The dyes were chosen since they generate distinctive, specific SERRS spectrum. However, since the dye labels have different absorbance maxima they will not all be in resonance with the same laser excitation frequency and this property can be exploited to produce a very sensitive and selective method for detecting each of these dyes within a mixture of the others using two different laser excitation frequencies.

Multiplexed assays with 6 dye labelled oligonucleotides using a single excitation source have also been carried out and show the difficulty of separating the spectra by eye. Faulds et al. (Analyst, 2008, 133, 1505-1512 (2008)) adopted a multivariate analysis approach where the whole of the SERRS spectrum is considered, rather than looking for specific discriminatory Raman bands. Using this approach the first multiplexed simultaneous detection of six different DNA sequences, corresponding to different strains of the E. coli bacterium, each labelled with a different commercially available dye label (ROX, HEX, FAM, TET, Cy3, or TAMRA) was reported. In this study, both exploratory discriminant analysis and supervised learning, by partial least squares regression, were used and the ability to discriminate whether a particular labelled oligonucleotide was present or absent in a mixture was achieved using partial least squares regression with very high sensitivity (0.98-1), specificity (0.98-1), accuracy (range 0.99-1), and precision (0.98-1).

In the above two multiplexed examples, the sample mixture was prepared using different types of labelled-oligonucleotides, all present at the same concentration. However, this will not be the case for many industrial and diagnostic applications, where some analytes may be present at relatively high concentrations and others at relatively low concentrations. In addition, many effective SERRS dyes share significant chemical similarity, leading to many shared features in their SERRS spectra and making them harder to resolve in mixtures. The difference in dye absorption cross-sections can also lead to a weak scattering dye being obscured by a strong scattering dye. Thus, for many practical applications, a method is required to provide both a high degree of multiplexing with each component identifiable over a range of concentrations and to enable the effective construction of a number of different multiplex combinations

### SUMMARY OF THE INVENTION

The present invention is based in part on a method for constructing effective combinations of dyes which can be used to detect various analytes which may be present over a concentration range in a sample and which can be discerned in a single wavelength multiplex assay.

According to a first aspect of the invention, there is provided a kit for use in a multiplex assay, the kit comprising a dye set consisting essentially of a plurality of dyes and an association of each dye to a reference concentration, wherein, using surface enhanced Raman spectroscopy (SERRS), each dye of the set is identifiable at better than 90% sensitivity and 90% specificity in the presence of any other dye of the set throughout a range of concentrations of each of the two dyes from 0.6 to 1.5 of the respective dye's reference concentration.

A kit according to the invention may be used for detecting different analytes in a sample, for example by attaching each dye to a ligand to form a dye-ligand conjugate, each ligand capable of binding to a specific analyte. The dyes may be used to detect analytes even when in the presence of another dye. This may be useful when detecting more than one disease in a sample from a patient. Furthermore, a kit according to the invention may provide a user with a higher level of confidence that an analyte will be detected because the dyes are identifiable in the presence of any other dye of the set across a range of concentrations. This may be important as a user may want to be confident that an analyte is detected even when a concentration of the dye is not precisely that of the reference concentration.

Preferably, each dye is identifiable at better than 95% sensitivity and/or 95% specificity, and more preferably, at better than 98% sensitivity and/or 97% specificity, in the presence of any other dye of the set throughout a range of concentrations of each of the two dyes from 0.6 to 1.5, and preferably 0.45 to 1.5 of the respective dye's reference concentration. Such high levels of sensitivity and specificity are desirable in medical diagnostics, wherein failure to identify an analyte or incorrect identification of an analyte may have serious repercussions.

It may be desirable to have different reference concentrations for the dyes of the set. In this way, the range of concentrations over which a dye can be identified may be increased by selecting lower reference concentrations of the dyes having more intense SERRS spectra for a specified excitation wavelength. A difference in the reference concentrations for any pair of dyes of the set may be less than 2 orders of magnitude and may be between 1×10⁻¹¹ Molar and 1×10⁻⁹ Molar and further optionally may be between 4×10⁻¹¹ Molar and 3×10⁻¹° Molar.

It will be understood that the term “the dye set consisting essentially of a plurality of dyes” means that the dye set does not comprise any other dyes that are essential for detecting analytes in the multiplex assay beyond the plurality of dyes. However, the dye set may comprise other substances such as water, spermine and colloid.

The dye set may comprise a mixture made of the plurality of dyes. Alternatively, each dye of the set may be housed separately.

The association of each dye with the reference concentration may be an association of each dye to a reference SERRS spectrum used in the multiplex assay for identifying the dye, the reference SERRS spectrum obtained with the dye present in a sample at the reference concentration. For example, the kit may comprise a library of such reference SERRS spectra or alternatively, the kit may comprise identification of a source of reference SERRS spectra that should be used to analyse the dyes. Alternatively, the kit may comprise a reference sample for each dye, each reference sample comprising a mixture including the dye at the reference concentration. In use, a user may obtain reference spectra using the reference samples, the reference spectra for use in identifying dyes in a sample, for example using a Direct Classical Least Squares (DCLS) analysis.

The dye set may be made of a plurality of dyes for example 5, 6, 7, 8, 9, 10 or more dyes. The dye set may comprise of at least six dyes from any one of the following lists:

### i) JOE, Rhodamine Green, ATTO520, BODIPY FL, BODIPY TMR-X, FAM, HEX, Cy3, Cy3.5, TAMRA and TYE563;

### ii) JOE, Rhodamine Green, FAM. HEX, DY549, Cy3, Cy3.5, ATTO488, MAX and TYE563;

iii) BODIPY530/550, BODIPY FL, BODIPY TMR-X, CY3.5, CY3, FAM, HEX, Rhodamine Green, TAMRA and TYE563.

The dye set may comprise CY3.5, CY3, FAM, HEX, Rhodamine Green and TYE. In one embodiment the dye set may consist of 10 of the following dyes: JOE, Rhodamine Green, ATTO520, BODIPY FL, BODIPY TMR-X, FAM, HEX, Cy3, Cy3.5, TAMRA and TYE563. In another embodiment, the dye set may consist of JOE, Rhodamine Green, FAM, HEX, DY549, Cy3, Cy3.5, ATTO488, MAX and TYE563. In a further embodiment, the dye set may consist of BODIPY530/550, BODIPY FL, BODIPY TMR-X, CY3.5, CY3, FAM, HEX, Rhodamine Green, TAMRA and TYE563.

However, in accordance with the teaching described herein, the skilful addressee is able to identify other sets of dyes which may be used.

According to a second aspect of the invention there is provided a kit for use in a multiplex assay comprising a dye set, the dye set consisting essentially of a plurality of dyes, each dye for use in identifying a separate analyte in a sample using surface enhanced resonant Raman spectroscopy when a concentration of the dye is less than 1×10⁻⁹ Molar, optionally, less than 5×10⁻¹⁰ Molar, and a concentration difference in the sample between the dye and any one of the other dye spans is at least 2×10⁻¹¹ Molar.

It will be understood that the use herein of the term “surface enhanced Raman spectroscopy (SERRS)” in connection with the invention it intended to include arrangements wherein there is an overlap in the absorption and plasmon resonance profiles although the centre of each profile occurs at a different wavelength. The centre of each profile may be offset by less than 100 nm.

According to a third aspect of the invention there is provided a kit for use in a multiplex assay, the kit comprising a dye set consisting essentially of a plurality of dyes, wherein, using surface enhanced Raman spectroscopy (SERRS), each dye of the set is distinguishable from any other dye of the set at better than 99% sensitivity and 99% specificity.

According to another aspect of the invention, there is provided use of a kit according to the first or second aspects of the invention for detecting one or more analytes present in a sample, using a single excitation wavelength.

According to yet another aspect of the invention, there is provided a method for conducting a multiplex assay on a sample, the method involving:
providing dye-ligand conjugates wherein each ligand is bound to a different dye and is specific to an analyte,
forming a mixture by mixing the dye-ligand conjugates with the sample in order to allow the dye-ligand conjugates to bind with any specific analyte present in the sample and removing unbound dye-ligand conjugates;
measuring a spectrum of the mixture using a transduction technique;
identifying which analyte(s) is/are present in the sample by comparison of the spectrum to a reference spectrum for each dye, the reference spectrum obtained from a sample in which the dye is at a reference concentration using the transduction technique, wherein each dye is identifiable at better than 90% sensitivity and 90% specificity in the presence of any other dye of the set throughout a range of concentrations of each of the two dyes of 0.6 to 1.5 of the respective dye's reference concentration.

Preferably, identifying which analyte(s) is/are present in the sample involves irradiating the dye-ligand conjugates with a single excitation wavelength.

A number of different types of analytes that can be identified may be greater than 5, for example 6, 7, 8, 9, 10 or more and the ligand may be a peptide, an oligonucleotide an antibody or a protein for example.

The transduction technique is preferably a Raman based spectroscopy technique such as surface enhanced resonant Raman spectroscopy. However the transduction technique may also be a fluorescence technique. The transduction technique may also involve a single excitation wavelength for example a Raman excitation wavelength at 532 nm.

The analyte concentration may be extracted over a dynamic range of at least 1 order of magnitude for example 2, 3 or 4 orders of magnitude.

According to another aspect of the invention there is provided a method of selecting X dyes among N dyes comprising generating SERRS Raman spectra for dye sets, each dye set comprising one or more dyes selected from the N dyes, and calculating a figure of merit indicative of a chance of identifying, from the SERRS Raman spectra, correctly as present the one or more dyes in each set and/or incorrectly as present the dyes absent from each set and selecting the X dyes based upon the calculated figure of merit.

X and N represent integers, wherein X is less than N.

In this way, a “best” set of dyes are selected based upon the figure of merit.

The dyes may include dye sets comprising two or more dyes. In this way, the dyes may be selected based upon a chance of correctly identifying a dye and/or incorrectly identifying an absent dye as present when each dye is in the presence of one or more other dyes. The selected dyes may therefore be particularly suitable for identifying analytes when more than one analyte is present in a sample.

The method may comprise establishing a reference concentration for each dye, wherein the SERRS spectra generated for each dye set are based upon the dye set comprising one or more major dyes and a minor dye, wherein a ratio of a concentration of the or each major dye relative to the major dye's reference concentration is greater than a ratio of a concentration of the minor dye relative to the minor dye's reference concentration. Establishing the reference concentration may be based upon a limit of concentration of the dye when present as a minor dye in a dye set at which a sensitivity of the minor dye is above a set performance criteria, for example, a reference concentration may be chosen for each dye such that no one dye has a limit of concentration above a defined level, such as above 0.6, and preferably, above 0.45, of the reference concentration. Any dye for which a reference concentration cannot be identified for which the dye meets this criteria may be removed as an unsuitable dye. A further selection of the dyes may be based upon a limit of concentration determined for the established reference concentration, although other performance criteria, such as specificity, for these dye sets may first be considered as selection criteria. Using different reference concentrations for different dyes may allow the use of a dye that generates a weaker SERRS signal to be used with a dye(s) that generates stronger SERRS signals through appropriate shifting of the reference concentrations.

The method may comprise, for each dye set, determining specificity and/or sensitivity at at least one concentration, and preferably, at a plurality of different concentrations, of the minor and/or major dye and selecting the X dyes comprises selecting the dyes based upon the determined specificity and/or sensitivity. It may not be sufficient to check the specificity and/or sensitivity just at the limits of concentration of the dyes of each set but it may be necessary to check each dye set across a range of concentrations.

The method may comprise selecting a subset of dyes based upon a figure of merit calculated for m-plex dye sets then selecting dyes from the subset based upon a figure of merit calculated for p-plex dye sets formed from combinations of the subset of dyes, wherein each m-plex dye set comprises fewer dyes than each p-plex dye set. For example, the m-plex dye sets may be simplex dye sets and the p-plex dye sets may be duplex dye sets. The processing required to screen the dyes may increase as higher order plex dye sets are analysed if the number of dyes from which the selection is to be made remains the same. Accordingly, reducing the number of dyes by screening the dyes first though analysis of lower plex dye sets may reduce the time it takes to select the X dyes from the N dyes.

A further method for reducing the processing time to select the X dyes is to use core mixtures (such as duplexes, triplexes and quadruplexes, etc) that are known to meet the necessary requirements as a starting point for selecting the dyes, the method comprising determining dyes to add to the established core mixture.

The SERRS spectra may be simulated from data indicative of the variability of SERRS spectra obtainable from each dye of the set. For example, the variability data may comprise SERRS spectra for each dye obtained experimentally under different conditions. These SERRS spectra are different to a reference SERRS spectrum that may be used for identifying the dye. The SERRS spectra for each dye set may be generated by randomly selecting SERRS spectra from the variability data and applying scaling of the intensity as appropriate for the concentrations. In the case of dye sets comprising more than one dye, it may be necessary to remove features of the spectra that are duplicated when two or more spectra are combined, such as background.

According to another aspect of the invention there is provided a data carrier having instructions stored thereon, which, when executed by a processor, cause the processor to carry out the method of selecting dyes in accordance with the aspects described above.

According to a further aspect of the invention there is provided a computer system programmed to carry out the method of selecting dyes in accordance with the aspects described above.

Preferably, identifying a dye in a dye set involves analysing the spectrum using a multivariate analysis technique, such as a method based on a Direct Classical Least Squares method.

According to yet another aspect of the invention, there is provided a dye set obtainable by the method for selecting X dyes among N dyes described above.

According to yet another aspect of the invention, there is provided a dye-ligand set wherein the plurality of dyes is obtainable by the method for selecting X dyes among N dyes described above.

According to yet another aspect of the invention, there is provided a method for detecting analytes in a sample and a method for conducting a multiplex assay on a sample, wherein the dye-ligand conjugates are made of dyes selected according to the method for selecting X dyes among N dyes described above.

### BRIEF DESCRIPTION OF THE DRAWINGS

Various aspects of the invention will now be described by way of example only and with reference to the accompanying drawings, of which:

FIG. 1 shows a flow diagram of a method for selecting a set of dyes.

FIG. 2 shows a flowchart illustrating a method for determining components present in a sample.

FIG. 3 shows a diagrammatic representation of a dye selection process in accordance with one embodiment of the invention.

FIG. 4 presents the individual false positive risk calculated for each dye absent in a known simulated spectrum using a terminated-direct-classical-least-squares fitting algorithm with a lack of fit of 15%.

FIG. 5 presents the calculated lowest detectable concentration of a minor dye in presence of a major dye in a dye-dye duplex, in order to achieve a true positive rate of 99%.

FIG. 6 presents the false positive rate calculated at the lowest detectable concentration achievable for a minor dye and a minor-dye/major-dye duplex.

FIG. 7 shows a protocol of a SERRS-multiplex-homogeneous-assay.

FIGS. 8a to 8n are diagrams showing the chemical structure of the dyes, and

FIG. 9 shows a system for carrying out a multiplex assay according to the invention.

### DETAILED DESCRIPTION OF THE INVENTION

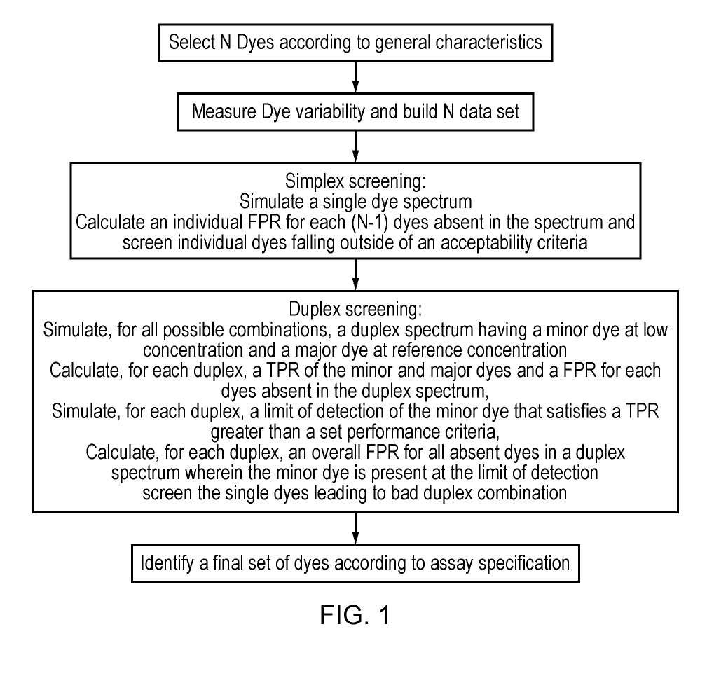

FIG. 1 shows steps in a method for selecting a set of dyes that can be used in combination for multiplex diagnostic applications. The method involves five steps.

The First step relates to the pre-selection of N dyes in a dye pool, where dyes are assessed based on the following criteria:
a) The dye signal should be stable, and unaffected by the presence of other dyes.
b) Dye signals should show a linear response with concentration following the Beer-Lambert law.
c) The dye SERRS spectral profile. The profile must be unique, and ideally dyes should show strong SERRS signals with a low fluorescent background. The best dyes present at least one discriminating spectral feature.
d) The dye chemical stability.
e) The dye dynamic range. The SERRS features of the dye should be detectable across a wide range of concentrations.
f) The dye chemical affinity. For example, the dye should present high chemical binding affinity to a SERRS surface.
g) The level of SERRS enhancement. For example, SERRS enhancement is related to the way a dye affects the aggregation of nanoparticles (size effect, electrostatic interactions). Unless already known, the level of SERRS enhancement is evaluated experimentally for each dye-nanoparticle complex.

The assessment of these criteria may be carried out by eye, for example by viewing SERRS spectra generated by the dyes, and any dye that clearly fails one or more of these criteria may not be included in the dye pool. This pre-selection of dyes removes dyes that are clearly inappropriate, reducing the processing required in subsequent steps.

The second step is to measure spectra encompassing signal variability for each of the N dyes in the dye pool at a reference concentration. In this embodiment, the reference concentration(s) of the dye in a reference sample is/are selected to be between 1×10⁻¹¹ Molar and 1×10⁻⁹ Molar. Dye spectrum variability is acquired according to a fractional factorial experimental design, and covers a plurality of variability parameters which may include: the operator (the person preparing and measuring the spectra), the batch of dye, the batch of colloid, the batch of spermine, preferably present as hydrochloride, and the time that has elapsed between sample preparation and measurement. For each dye, a series of SERRS spectra are measured along with a number of “blank” reference spectra. There is no lower or upper limit on the number of spectra measured for each dye, but each set of variability spectra should encompass factors affecting variation in the dye signal.

In some cases, the data collected during this step may indicate that the dye should not be included in the dye pool, for example, because it fails to meet the criteria for pre-selection outlined above in the First Step. This can occur if pre-selection choices are based upon a smaller and less representative set of spectra than are collected for the dye variability step.

The SERRS spectra of the variability data are filtered to remove spectra where the dye signal is either significantly weaker or stronger than the average. This is intended to remove outlying spectra which should not be part of the set.

The third step referred to as Simplex Screening, estimates the risk of a false positive result when analysing a SERRS spectrum of a single dye. In other words, when analysing the SERRS spectrum of a single dye, the risk of identifying another (absent) dye.

The true positive and false positive rates are defined as follows. When considering the analysis of a sample in which dye A is present and dye B is absent, if for example the sample is analysed one thousand times, and if the analysis detects the presence of dye A in 995 cases, the estimated true positive rate (TPR) of dye A will be 99.5%. Similarly, if another dye, B, absent in the sample is incorrectly detected by the analysis in 10 cases, the estimated false positive rate (FPR) of dye B will be 1%. The TPR corresponds to sensitivity, and the FPR corresponds to selectivity.

The false positive rate is simulated for each individual dye by carrying out the following steps a number of times in order to obtain a statistically acceptable measure of a dye's false positive rate.

A reference spectrum is randomly selected for each dye from the variability data and a spectrum is simulated for the “present” dye by randomly selecting a further spectrum, different to the reference spectrum chosen for that dye, from the variability data.

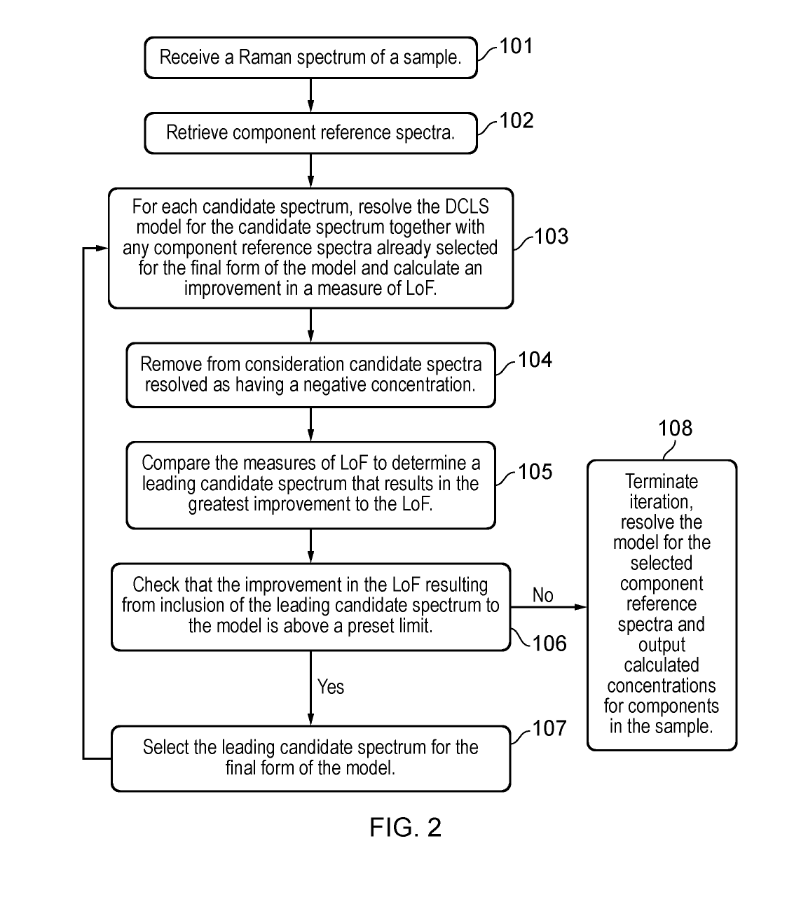

The simulated spectrum is analysed using the algorithm described below with reference to FIG. 2 and the selected reference spectra. Any false positive results for each of the absent (N−1) dyes are noted.

These steps are then repeated an appropriate number of times to obtain a statistically significant measure of the estimated false positive rates. Any false positive rates significantly above a threshold would indicate that the corresponding pair of dyes (one present, one absent but falsely detected) represent a false positive risk, and hence are not a good pairing to use in an assay.

Alternatively, an extended Simplex Screening simulation can be performed. This is essentially the same as the basic Simplex Screening simulation described above, but with one modification. In the basic form of the simulation, it is assumed that all of the dyes in the dye pool could be present in the sample, and the spectrum is analysed accordingly. However, this presents a small risk of “hiding” some possible false positive risks. For example in the instance where a dye A is present, and dyes B and C both have a risk of being incorrectly “detected” under these circumstances. If dye B has a slightly better fit than dye C, it will be chosen preferentially over dye C every time, and thus potentially masks the risk between dyes A and C. So in this variation of the simulation, the analysis is repeated several times, covering every possible pairing in a two dye system. For example, when dye A is present, the data is analysed assuming only that dyes A & B could be present, then the analysis is repeated assuming that only dyes A & C could be present, then only A & D . . . etc. This approach helps find additional False Positive risks that might otherwise have been missed.

Following simplex screening, dyes that are associated with poor specificity results may be considered for removal from the dye pool. However, removal of any one dye from the pool may eliminate the need to consider removal of other dyes from the pool. For example, if dye A shows poor specificity when analysed with dye B also potentially present in the sample, then removal of either one if these dyes may eliminate the need to also remove the other, provided the other dye does not have additional specificity issues with a third dye. Often, there are multiple viable options for removing a small number of dyes from the pool in order to leave a smaller sized dye pool with no significant specificity issues.

The fourth step, referred to as Multiplex Screening, estimates the risk of a false negative result when analysing a SERRS spectrum of a sample containing two or more dyes, and also checks that the estimated risk of a false positive result is not significantly higher than for separate simplex samples of the dyes present in the multiplex sample.

Multiplex Screening is illustrated by discussing the Duplex case. Considering a duplex having a “minor” dye at low concentration relative to its reference concentration in presence of a “major” dye a set concentration at or above its reference concentration, this step of the method calculates the minimum concentration of the minor dye that is sufficient to achieve a set performance criteria, for example TPR above a required limit, for example >99%. The minimum concentration of the minor dye that meets the performance criteria is defined as the lowest detectable concentration (LDC) of the minor dye.

To do so, the Raman spectrum of a mixture containing a (fixed) high concentration of dye A+a low concentration of dye B is simulated by combining spectra randomly selected from the variability data for dye A and B, the spectra scaled as appropriate for the concentrations. An appropriately-scaled blank spectrum (also chosen randomly from the variability data) is either added or subtracted as appropriate, to keep the overall “blank contribution” to the simulated spectrum appropriate. As with the simplex screening, a reference spectrum is selected for each candidate dye and the simulated spectrum analysed using the DCLS algorithm as described below with reference to FIG. 2.

These steps are then repeated an appropriate number of times to obtain a statistically significant measure of the estimated true positive and false positive rates.

Assuming that the required performance criterion is not met, the concentration of dye B is increased by a small amount (for example 1%), but the concentration of dye A is kept the same. The method is repeated until the TPR meets its required limit. The corresponding concentration is the lowest concentration of dye B which can reliably be detected if dye A is present at the set concentration at or above the reference concentration.

The overall FPR is estimated (for all absent dyes) for each duplex at a range of concentrations of the minor dye, in order to confirm that the duplex combination does not have a significantly higher False Positive risk than simplex samples containing the dyes that make up the duplex.

This approach can be extended to higher-order multiplex levels, for example triplex or four-plex. In such cases, when sensitivity is the main property of interest, a suitable approach is to simulate samples with multiple major dyes and a single minor dye. For example, a triplex simulation may contain two major dyes with concentrations at or above their respective reference concentrations, and a single minor dye below its reference concentration.

Multiplex combinations associated with poor performance (a high LDC and/or or high FPR) are then identified. The individual dyes associated with the multiplex may be considered for removal from the dye pool. Alternatively, if the poor performance of the multiplex is due to False Positives, the dye or dyes which are incorrectly identified as present (the False Positive dye or dyes) may be considered for removal from the dye pool. As is the case with the Simplex Simulation, removing any one dye from the pool may eliminate the need to remove other dyes, and there may be more than one way to eliminate all poor-performing multiplex combinations.

In general, duplex screening is performed before triplex screening, which in turn is conducted before higher-order multiplex screening. This allows the results from the lower-order multiplex simulations to reduce the number of dyes in the dye pool that is used in the higher-order multiplex simulations, reducing the simulation complexity.

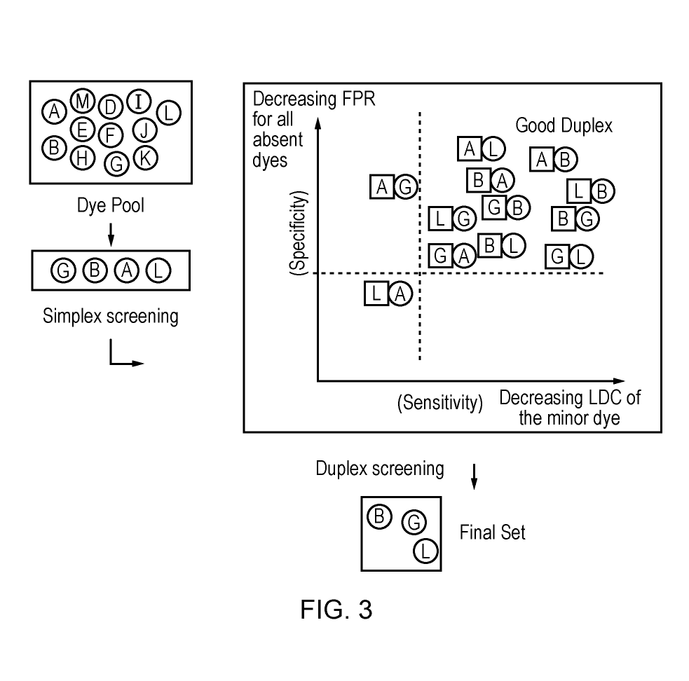

FIG. 3 shows a representation of duplex classification according to the overall FPR and LDC of the minor dye present in each duplex (the conjugated duplex is not shown for clarity). The major dye is represented by a rectangular box and the minor dye is represented by a circle. The single dyes leading to frequent bad combinations (i.e. high overall FPR and/or poor LDC) are removed from the pool of dyes. For example in FIG. 3, dye A has been identified to lead to a majority of poor performing duplexes.

During multiplex screening, if it is found that different dyes in the dye pool have very disparate lowest detectable concentration values, then the dye reference concentrations may be adjusted and the process re-started. For example, if dye A has a limit of concentration of 5% of the reference concentration whereas dye B has a limit of concentration of 30% of the reference concentration, the reference concentration for dye A may be increased and/or the reference concentration for dye B reduced. This may require the gathering of new variability data at these new reference concentrations.

The fifth step involves the selection of X dyes from the remaining N dyes in the dye pool based on data generated in the preceding steps. If there are more dyes remaining in the dye pool than needed for a specific application (i.e. N is greater than X), further selection is required in order to identify which set of dyes would achieve the best result. Depending on the application it could be a choice between better sensitivity, specificity and some combination of the two.

Referring to FIG. 2, the Direct Classical Least Squares technique for analysing the simulated spectra models the simulated spectral data X in terms of a set of K known component reference spectra Sₖ each having I data points. Component concentrations, Cₖ, for each component reference spectrum are determined by minimising the sum of the squared deviations of the spectral data from the reconstructed model,

$${{Σ}_{i}^{I} [ {X}_{i} - {Σ}_{k}^{K} {C}_{k} {S}_{ki} ]}^{2} \qquad ( 1 )$$

where i represents the spectral frequency index. This results in a series of linear equations which are solved directly by matrix inversion for the component concentrations Cₖ.

An iterative process is carried out in which Equation (1) is resolved for each candidate dye using the selected reference spectrum together, steps 103 to 108.

In step 103, for each candidate dye, equation (1) is minimised for the dye's reference spectrum together with any dye reference spectra that have already been selected in a previous iteration. A measure of goodness of fit is calculated for the resolved components relative to the simulated spectrum.

The measure of goodness of fit can be a measure of lack of fit (LoF) given by:—

$$LoF = \sqrt{\frac{{{Σ}_{i = 1}^{I} [ {x}_{i} - {Σ}_{k = 1}^{K} {c}_{k} {s}_{ki} ]}^{2}}{{Σ}_{i = 1}^{I} {x}_{i}^{2}}} \qquad ( 2 )$$

This measure of lack of fit is compared to a previous measure of LoF calculated for the selected dye reference spectra before the addition of the candidate dye reference spectrum to determine an improvement to the measure of LoF resulting from the addition.

The improvement in the LoF, Lₗₚᵣ, is calculated as a proportional improvement in the LoF:—

$${L}_{Ipr} = \frac{{L}_{old} - {L}_{new}}{{L}_{old}} \qquad ( 3 )$$

where L_{old} is the LoF value calculated for the selected dye reference spectra before the inclusion of the candidate dye reference spectrum and L_{new} is the LoF value calculated for the selected dye reference spectra including the candidate dye reference spectrum.

In step 104, the candidate dye reference spectra resolved as having a negative concentration are removed from further consideration in the iteration (but not subsequent iterations).

In step 105, the improvements in the LoF, Lₗₚᵣ, for the remaining candidate dye reference spectra are compared and the candidate dye reference spectrum associated with the greatest improvement in the LoF becomes the leading candidate dye reference spectrum for inclusion in the final form of the model.

A check 106 is made to determine whether the improvement in the LoF resulting from addition of the leading candidate dye reference spectrum is above a preset limit. If the improvement to the LoF, Lₗₚᵣ, for the leading candidate dye reference spectrum is above the preset limit, it is selected 107 as a dye reference spectrum that is present in the final form of the model. The process 103 to 107 is then repeated for the remaining unselected dye reference spectra.

If the improvement to the LoF, Lₗₚᵣ, for the leading dye reference spectrum is below the preset limit, then the method is terminated and the final form of the model, comprising the model resolved for the dye reference spectra selected up to that point, is output. The final form of the model will typically comprise a subset of the set of predetermined dye reference spectra, these spectra being those of most significance as measured by lack of fit.

A determination can be made of components present in the sample based upon whether the reference spectrum corresponding to a dye is included in the final form of the model.

Further details regarding the above method and of the preferred apparatus for conducting this method can be found in UK patent “Spectroscopic apparatus and methods for determining components present in a sample” application number EP11250530.0, filed on 16 May 2011.

A dye set identified using the above method may be supplied for use in a multiplex assay. The dye set should be supplied in association with a reference concentration for each dye such that the sample comprising the dyes is analysed using reference spectra obtained at the reference concentration. Such an association may be supply of the reference spectra themselves, information on where such reference spectra may be obtained, such as a website, etc, supply of reference samples wherein the dyes are at the reference concentration, a list of reference concentrations at which reference spectra should be obtained or/and supply of the dye set for use with a particular system, wherein the system comprises a library of reference spectra obtained at the reference concentrations.

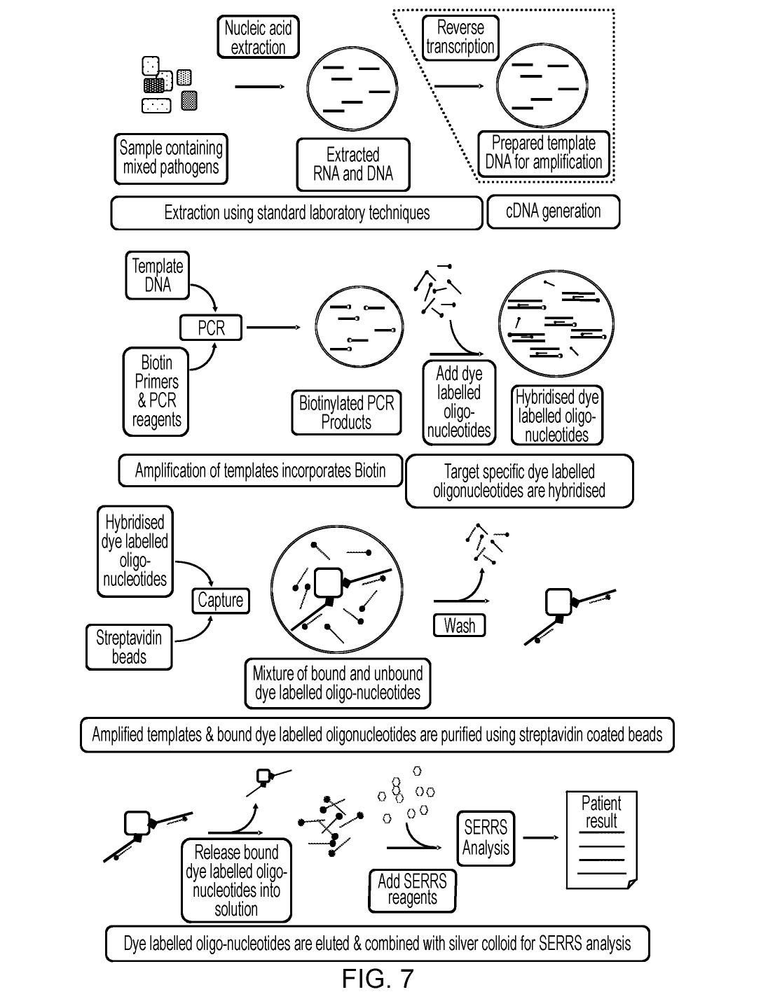

Now referring to FIG. 7, a method of using the dyes in a multiplex assay is described. A sample is obtained from a patient, the sample potentially containing a mix of pathogens. Using standard techniques, the RNA and DNA are extracted from the sample and template DNA obtained using reverse transcription where needed. The template DNA is amplified using a polymerase chain reaction (PCR) to a concentration roughly that of a reference concentration. Amplification of the DNA to such a concentration is achieved by appropriate selection of the PCR conditions, which are determined empirically. As part of the PCR process, biotinylated primers are added to the mixture such that the PCR process results in biotinylated products that can be captured later in the process using streptavidin beads.

The dyes are attached to oligonucleotide sequences that are complimentary to DNA sequences found in the pathogens to be identified. These dye labelled oligonucleotides are added to the biotinylated PCR products such that the labelled oligonucleotides hybridise to any complimentary sequences that are present.

Streptavidin beads are then added such that the biotinylated products attach to the beads whist leaving the dye labelled oligonucleotides that have not hybridised to complimentary sequences unattached. These unattached dye labelled oligonucleotides can then be washed away

The remaining dye labelled oligonucleotides are released from the streptavidin beads into a solution using an elution process, the solution comprising SERRS reagents for use in analysis. A SERRS spectrum of the solution is obtained and the spectrum is analysed using the technique described with reference to FIG. 2 to identify the dyes that are present in the solution. Determining the dyes that are present allows one to determine the DNA products that were present in the amplified PCR product and therefore, what pathogens were present in the original sample. A report is generated listing the pathogens that have been determined as present in the patient's sample. A medical professional can then use the result to diagnose and treat the patient.

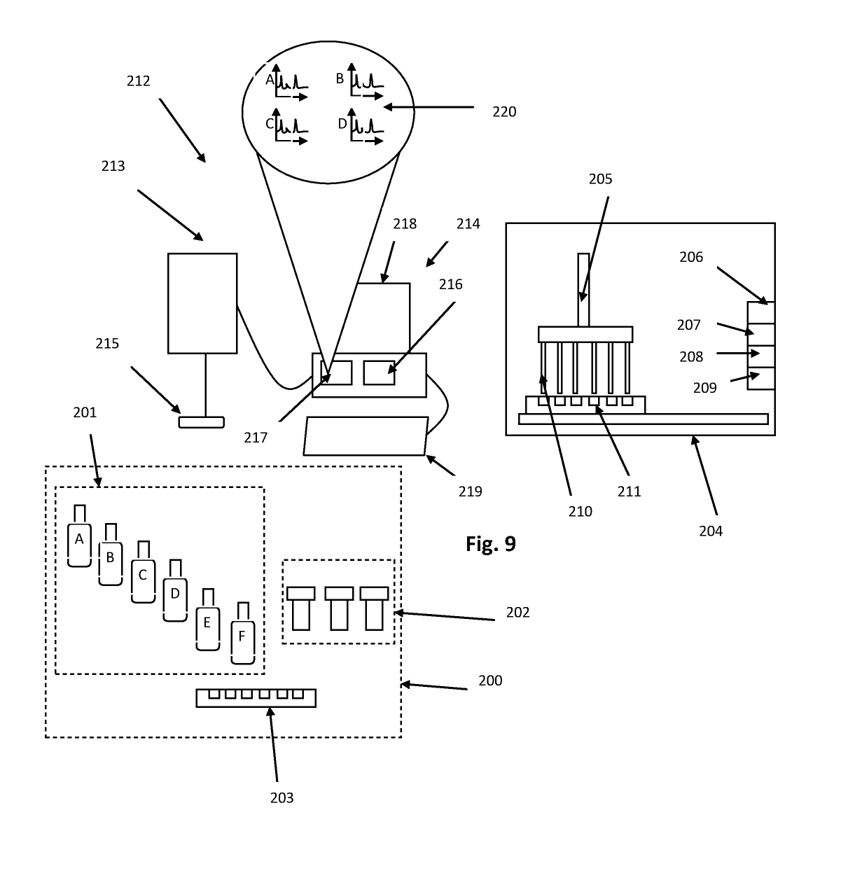

In order to carry out the above method, the dye kit may provided as part of a system, as shown in FIG. 9. The system comprises a kit 200 comprising a plurality of vials 201, each vial containing a dye labelled oligonucleotide complimentary to a specified target, a PCR kit 202 comprising primers and reagents for PCR and a micro-plate 203 comprising wells in which the PCR product containing the dye labelled oligonucleotides can be prepared. Other substances may be provided in the kit, such as magnetic beads, wash buffers, elution buffer and SERRS reagents, for use in the sample processor.

The kit 200 is a consumable and, as such, can be supplied to the consumer, as and when required. Furthermore, different kits for identifying different targets can be used. A kit may be provided for identifying causative agents of gastroenteritis. For example, the kit may be used in an assay to detect two or more of the bacterial targets ETEC, EPEC, VTEC, Salmonella, S. enterica, Campylobacter, Shigella, C. difficile A, C. difficile B and Yersinia. A kit may be provided for identifying two or more viral targets, such as one or more of Norovirus G1, Norovirus G2, Adenovirus, Rotavirus, Sapovirus and Astrovirus. A kit may be provided for identifying causative agents of fungal infections. For example, such a fungal kit may be used in an assay to identify two or more of A. fumigates, A. glaucus, A. flavus, A. terreus, A. Niger, A. ustus, A. candidus, A. versicolor. A kit may be provided for identifying causative agents of cerebrospinal fluid (CSF) viral infections. For example, the kit may used to identify two or more of the targets Herpes simplex virus 1, Herpes simplex virus 2, Varicella-zoster virus, Epstein-Barr virus, Cytomegalovirus, Enterovirus and poliovirus, John-Cunningham virus, Parechovirus. An alternative kit may be provided to detect two or more of the Candida species, such as two or more of C albicans, C. parasilosis, C. tropicalis, C. viswanthii, C. guilliermondii, C inconspicua, C. lustaniae, C. dubliniensis, C. kefyr, C. famata, C. krusei and C. glabrata. In each example, the same dyes may be used, with each dye attached to an oligonucleotide sequence that hybridises to a corresponding sequence on the target in the amplified PCR product.

The system further comprises a sample processor 204 for automatically carrying out steps of attaching the hybridised dye labelled oligonucleotides-PCR product complex to the magnetic beads, introducing a washing buffer to wash away the excess dye labelled oligonucleotides that are not attached to the target, introducing an elution buffer to detach the hybridized dye labelled oligonucleotides from the magnetic beads and combining with the SERRS reagents. This may be carried out by a robot arm 205 that controls a plurality of pipettes 210 to transfer a set volume of products contained in the wells of microplate 203, inserted into the sample processor 204 by the user to a further microplate 211 to which the magnetic beads, wash buffer, elution buffer and SERRS reagents can be added. The sample processor 204 comprises a number of reservoirs containing the magnetic beads, wash buffers, elution buffers and SERRS reagents. In the example, only four reservoirs are shown 206, 207, 208 and 209, but more than four reservoirs are preferably at least for the reason that there are a number of SERRS regents, each of which is kept in a separate reservoir. The robot arm can control the pipettes to take a set volume of these solutions when required.

The system further comprises a spectrometer 212 comprising a Raman spectrometer 213 for scanning a sample 215, the spectrometer connected to a computer 214. The computer comprises a processor 216, memory 217, a display 218 and an input device, such as a keyboard 219. Stored in memory is a set of Raman reference spectra 220 to be used in the analysis of the Raman spectrum obtained from the sample. The memory associates each Raman spectrum to a target for each different kit that may be used in the system. For example, the same dye may be associated with different targets for different kits.

In use, through appropriate inputs, a user identifies to the computer 214 the kit being used and the computer analyses the Raman spectrum of the sample using the reference spectra 220 for the dyes associated with this kit. The computer 214 can then output the targets that are deemed present in the sample based upon whether the dye associated with this target has been identified. Accordingly, in some sense, the Raman spectrum is sent to the computer but it may not be possible to decode the Raman spectrum into identified targets unless the correct reference spectra are used.

Because the user has identified the kit used to generate the Raman spectrum and the memory has stored therein reference spectra obtained at the required concentrations of the dyes, the computer can decode the Raman spectrum and provide an interpretation of the results, i.e. a list of all targets with detected and undetected stated alongside. Because the dyes and reference spectra have been selected such that, when using those reference spectra, any one of the dyes can be detected in the presence of any other one of the dyes across a range of concentrations around the reference concentration, multiple targets can be detected by the system, even if the targets are not present at the reference concentration. Without knowledge of the keys, i.e. reference spectra, to use to decode the Raman spectrum, the system may not be able to provide the technical operation of identifying targets in the sample.

The reference spectra are obtained from calibration plates comprising the dyes at the reference concentration. The user or a service engineer uses the Raman spectrometer 212 to obtain a Raman spectrum from each plate and these spectra are stored as reference in memory 217. These reference spectra may be updated at regular intervals to take into the performance of the Raman spectrometer.

As an alternative to the above, a solid assay may be performed using an array of dye-ligand spots bound on a SERRS active substrate, such as Klarite®. In this case a suitable set of dyes may be selected in order to provide surface enhancement from Klarite.

Use of a set of dyes selected in the manner described above ensures that there is a high level of confidence that a pathogen will be correctly identified as present or absent given that the exact concentration of the amplified DNA of the pathogen may not be exactly that of the desired reference concentration and, in particular, that the presence of the dye will not be masked by one or more other dyes that are also present.

It will be understood that the above multiplex assay technique is not limited to the identification of pathogens but other could be used to identify other organic matter.

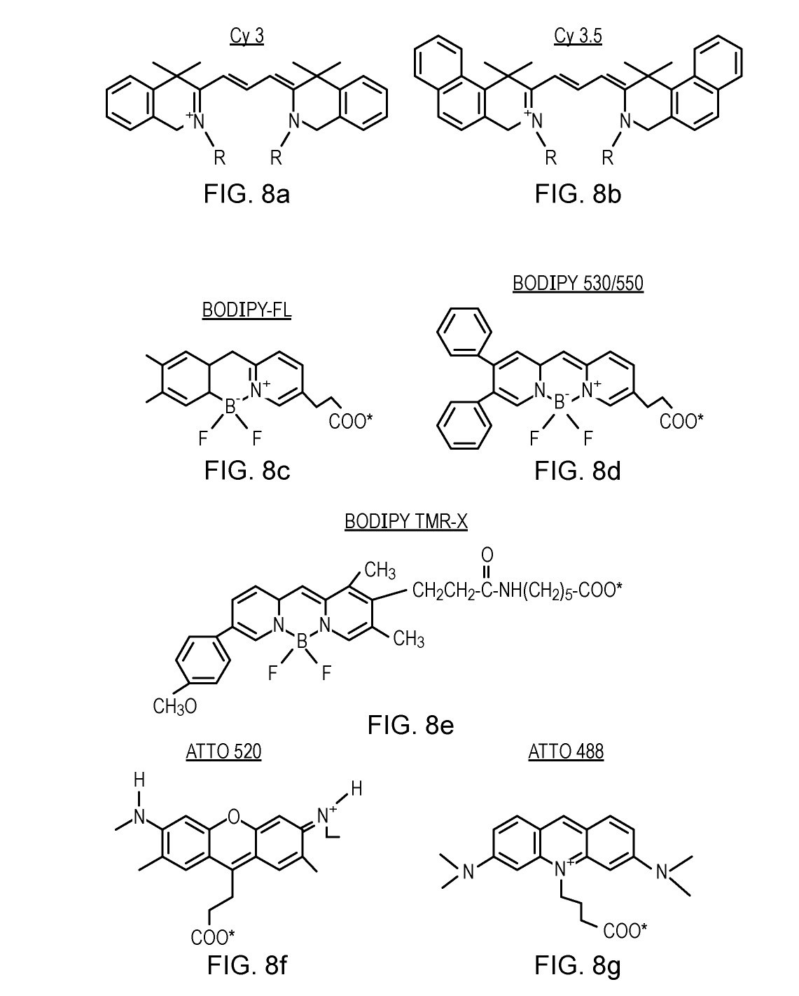

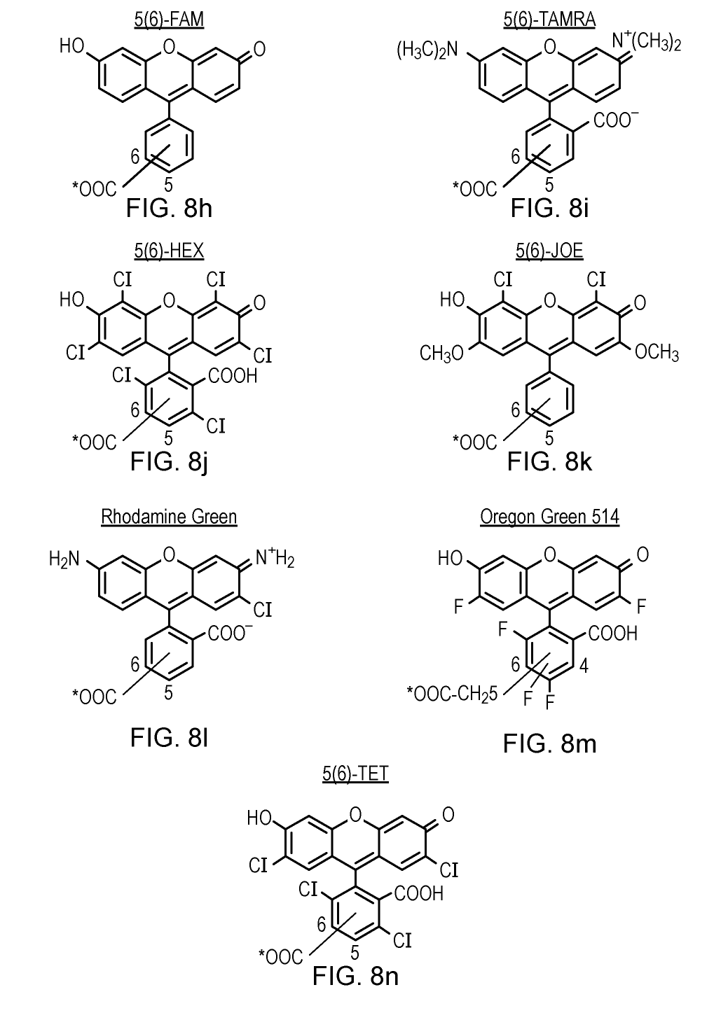

The next section presents as an example, the selection of 10 dyes for a SERRS diagnostic application from a pool initially containing 15 dyes. The 15 dyes in the pool provided for the experiment are: ATTO488, ATTO520, BODIPY 530/550, BODIPY FL, BODIPY TMR-X, CY3.5, CY3, FAM, HEX, JOE, MAX, Rhodamine Green, TAMRA, TET and TYE563. The chemical structures for these dyes are shown in FIGS. 8a to 8p.

Review of the dye variability data collected in the Second Step of the process indicated that ATTO488 and BODIPY 530/550 were unsuitable for this application, due to a larger-than-desired level of variability in the dye spectrum signal. They were therefore removed from the dye pool, leaving 13 dyes in the pool.

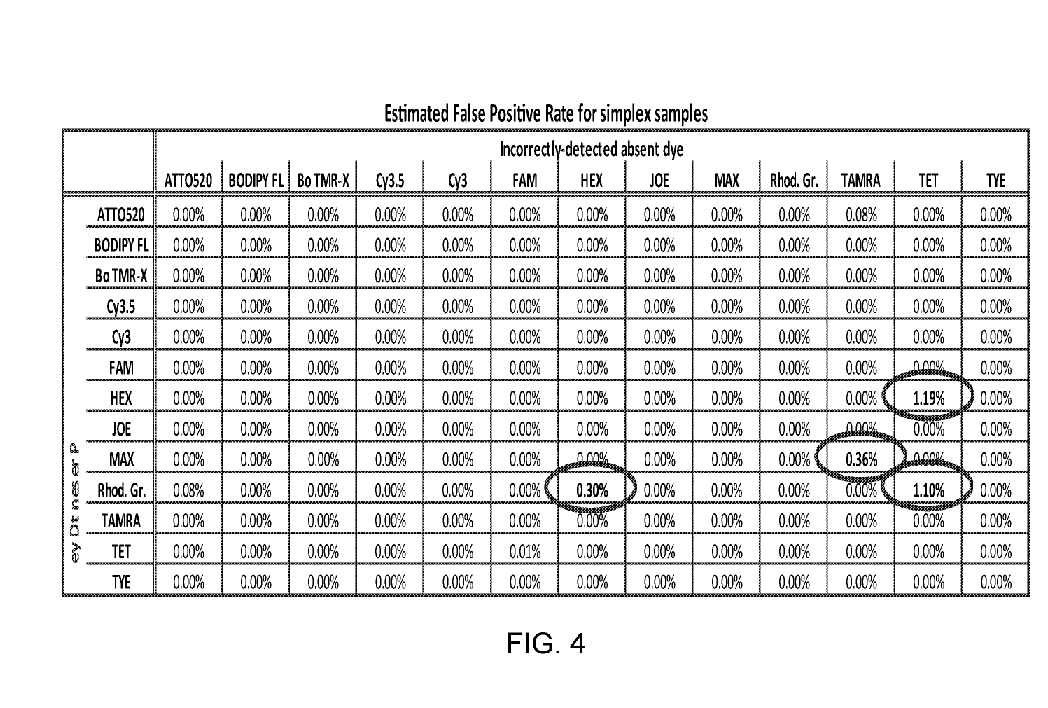

An extended Simplex Screening for False Positive risks was performed as described above (the Third Step), producing the results shown in FIG. 4. The most significant False Positive risks are circled.

Each value in the table corresponds to the estimated rate of incorrectly detecting a dye that is absent (in other words, obtaining a False Positive). For example, looking at FIG. 4, the estimated rate for falsely detecting TET in a spectrum containing only HEX is 1.19%, whereas the estimated rate for falsely detecting TAMRA in a spectrum containing ATTO520 is just 0.08%.

The most significant False Positive risks identified are between HEX and TET, and Rhodamine Green and TET. Other False Positive risks (for example, between MAX and TAMRA) are considered to be at or below an acceptable level for this application. The identified risks can be avoided by removing TET from the dye pool, or by removing both HEX and Rhodamine Green from the dye pool. In this case the decision was made to remove TET because this retains more dyes in the dye pool for use in subsequent steps of the process.

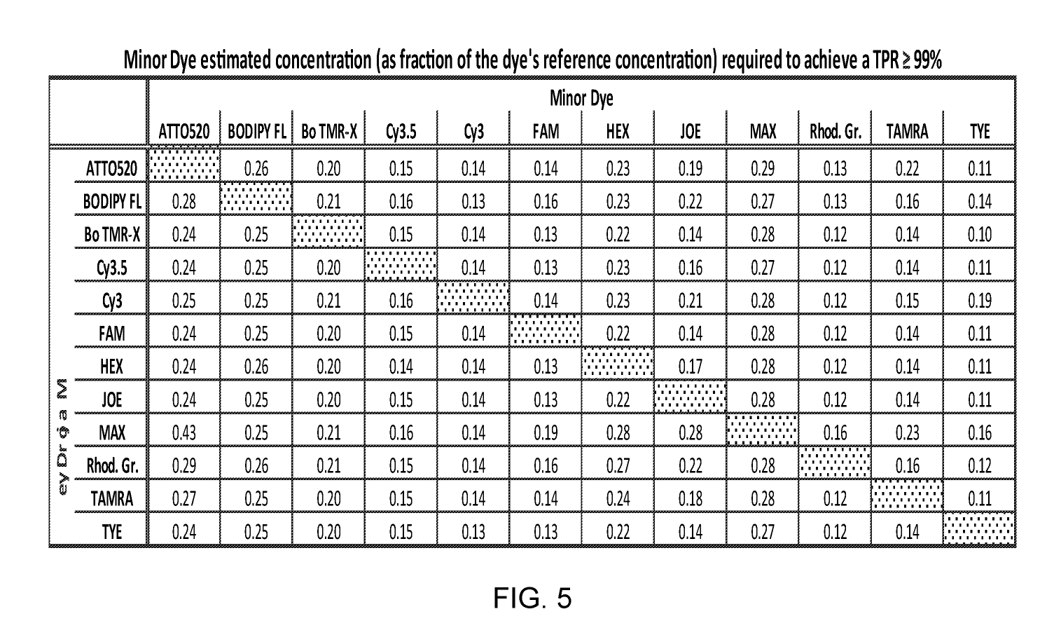

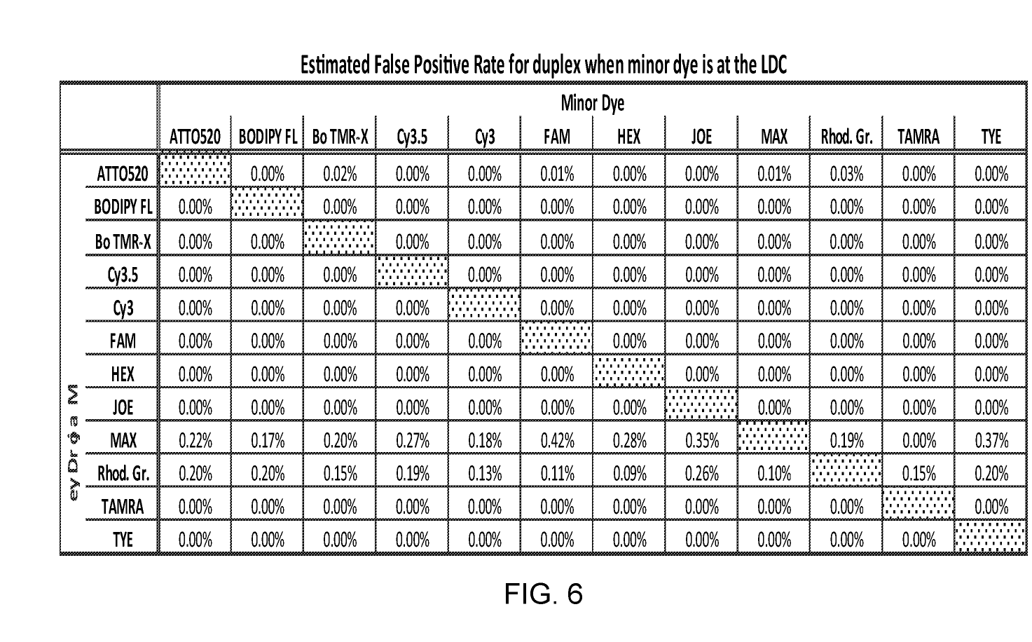

The results of a Duplex Screening (the Fourth Step) are shown in FIGS. 5 and 6. FIG. 5 shows the estimated lowest detectable concentration at which a True Positive Rate (sensitivity) of 99% is achieved for each duplex combination. For example, looking at the ATTO520/CY3 duplex, where ATTO520 is the major dye and present at reference concentration, and CY3 the minor dye and present below reference concentration, the lowest concentration at which CY3 is estimated to be detectable in 99% of cases is 0.14 times the CY3 reference concentration. These results suggest that the Lowest Detectable Concentration (LDC) is predominantly dependent on the identity of the minor dye, and only somewhat dependent upon the major dye it is present with, although some specific exceptions occur.

The duplex simulation also estimates the overall FPR (for all absent dyes) for each duplex when the minor dye is present at the corresponding LDC; these results are shown in FIG. 6.

Examining FIG. 5, we note that the duplex with the highest (worst) estimated LDC is ATTO520 in MAX, where a minor dye concentration of 0.43 times reference concentration is required. This is significantly higher than for any other duplex pairing. As there are more dyes in the pool than required for the application, we are able to avoid this pairing by removing (at least) one of these dyes. Examining FIG. 6 shows that the presence of MAX as the major dye is also associated with the highest (worst) False Positive Rates. Consequently, it is more favourable to remove MAX than ATTO520 in this instance.

This leaves 11 dyes in the pool (the original 15, minus ATTO488, BODIPY 530/550, TET and MAX), whereas only 10 dyes are required for the application. The values in FIGS. 4 to 6 can be used (the Fifth Step) to decide which of the remaining dyes to drop to arrive at the final 10-dye set. For example, if Specificity is of paramount importance, it would be best to consider removal of Rhodamine Green or HEX as this combination is associated with the highest False Positive risk. Alternatively, if Sensitivity is the priority, it may be more appropriate to consider removal of ATTO520 (which is the most challenging dye to detect as a minor dye). For this application the estimated performance for the remaining 11 dyes is considered acceptable, so the choice could also be based on additional factors such as the dye's performance in any steps of the diagnostic application upstream of the SERRS measurement, reliability of material supply, etc.

Below is an example of a dye set, including the dyes and the dye's reference concentration, according to one embodiment of the invention.

| Dye | Ref. Concentration |
|---|---|
| ATTO520 | 7.0 × 10⁻¹¹ |
| BODIPY FL | 1.5 × 10⁻¹⁰ |
| Cy3.5 | 2.2 × 10⁻¹⁰ |
| Cy3 | 1.4 × 10⁻¹⁰ |
| FAM | 1.2 × 10⁻¹⁰ |
| HEX | 7.0 × 10⁻¹¹ |
| JOE | 7.0 × 10⁻¹¹ |
| Rhodamine Green | 6.0 × 10⁻¹¹ |
| TAMRA | 1.7 × 10⁻¹⁰ |
| TYE | 9.0 × 10⁻¹¹ |

The dye sets presented above can be used to perform multiplex diagnostic assays as described above with reference to FIG. 7.

A skilled person will appreciate that variations of the disclosed arrangements are possible without departing from the invention. For example, although the description of the method is based on the selection of 10 dye candidates among a dye pool containing 15 dye candidates, the nature of the dye candidates is not limited to these 15 dyes and could include other dyes such as for example ATTO550, DY549, TEX and Oregon Green. Accordingly the above description of the specific embodiment is made by way of example only and not for the purpose of limitation. It will be clear to the skilled person that minor modifications may be made without significant changes to the operation described.

1. An evaluation unit comprising memory having stored thereon a reference spectrum for each dye of a set of dyes used in a multiplex assay, the reference spectrum obtained with the dye present in a sample at a reference concentration and a processor arranged to carry out the steps of:—
a) receiving a Raman spectrum generated using surface enhanced Raman spectroscopy of a sample;
b) modeling the Raman spectrum using the reference spectra to identify one or more of the dyes of the set present in the sample; and
c) generating an output based upon the dyes identified as present in the sample,
wherein the set of dyes is such that each dye can be identified though the modeling of step (b) at better than 90% sensitivity and 90% specificity in the presence of any other dye of the set throughout a range of concentrations of each of the two dyes from 0.6 to 1.5 of the respective dye's reference concentration.

2. An evaluation unit according to claim 1, wherein the memory has stored thereon, for each of a plurality of multiplex assays, a list of dyes used in that multiplex assay and reference spectra for the dyes, the processor arranged to receive an identifier identifying one of the plurality of multiplex assays used in producing the Raman spectrum and to model the Raman spectrum using the reference spectra of the dyes identified in memory as used in the identified multiplex assay.

3. An evaluation system according to claim 1, wherein the memory has stored therein targets associated with the dyes, the output comprising a report on the targets that are detected as present based upon whether the dyes associated with these targets are identified as present in the sample.

4. A system comprising an evaluation unit according to claim 1 and a spectroscopy apparatus, wherein the spectroscopy apparatus is arranged to send Raman spectra to the evaluation unit for analysis.

5. A kit for use in a multiplex assay, the kit comprising a dye set consisting essentially of a plurality of dyes and an association of each dye to a reference concentration, wherein, using surface enhanced resonant Raman spectroscopy, each dye of the set is identifiable at better than 90% sensitivity and 90% specificity in the presence of any other dye of the set throughout a range of concentrations of each of the two dyes from 0.6 to 1.5 of the respective dye's reference concentration.

6. A kit according to claim 5, wherein a difference in reference concentrations for any pair of dyes of the set is less than 2 orders of magnitude.

7. A kit according to claim 6, wherein a difference in reference concentrations for any pair of dyes of the set is between 1×10⁻¹¹ Molar and 1×10⁻⁹ Molar.

8. A kit according to claim 7, wherein a difference in reference concentrations for any pair of dyes of the set is between 4×10⁻¹¹ Molar and 3×10⁻¹° Molar.

9. A kit according to claim 1, wherein the association of each dye with the reference concentration is an association of each dye to a reference SERRS spectrum used in the multiplex assay for identifying the dye, the reference SERRS spectrum obtained with the dye present in a sample at the reference concentration.

10. A kit as claimed in claim 5, comprising a reference sample for each dye, each reference sample comprising a mixture including the dye at the reference concentration.

11. A kit as claimed in claim 5, comprising at least six dyes from any one of the following lists:
i) JOE, Rhodamine Green, ATTO520, BODIPY FL, BODIPY TMR-X, FAM, HEX, Cy3, Cy3.5, TAMRA and TYE563;
ii) JOE, Rhodamine Green, FAM, HEX, DY549, Cy3, Cy3.5, ATTO488, MAX and TYE563;
iii) BODIPY530/550, BODIPY FL, BODIPY TMR-X, CY3.5, CY3, FAM, HEX, Rhodamine Green, TAMRA and TYE563.

12. A kit as claimed in claim 5, wherein the dye set comprises CY3.5, CY3, FAM, HEX, Rhodamine Green and TYE563.

13. A kit as claimed in claim 5, wherein the dye set comprises CY3, TAMRA, HEX, Rhodamine Green, JOE and ATTO520.

14. A kit according to claim 5, wherein the dye set consists of 10 of the following dyes: JOE, Rhodamine Green, ATTO520, BODIPY FL, BODIPY TMR-X, FAM, HEX, Cy3, Cy3.5, TAMRA and TYE563.

15. A kit for use in a multiplex assay comprising a dye set, the dye set consisting essentially of a plurality of dyes, each dye for use in identifying a separate analyte in a sample using surface enhanced resonant Raman spectroscopy when a concentration of the dye is less than 1×10⁻⁹ Molar, optionally, less than 5×10⁻¹⁰ Molar, and a concentration difference in the sample between the dye and any one of the other dyes spans is at least 2×10⁻¹¹ Molar.

16. A kit as claimed in claim 5, wherein each dye forms part of a dye-ligand conjugate, each ligand being capable of binding to a specific analyte.

17. A kit according to claim 5 for detecting one or more analytes present in a sample using a single excitation wavelength.

18. A system for carrying out a multiplex assay comprising a dye kit according to claim 5, a kit for washing away unattached dye labels and a PCR kit comprising the primers and reagents for carrying out PCR.

19. A system according to claim 18, further comprising a sample processor in which a wash can be carried out.

20. A system according to claim 18, further comprising a spectrometer.

21. A system according to claim 18, further comprising at least one calibration plate comprising at least one reference sample for obtaining at least one reference spectrum to be used in the analyses of a SERRS spectrum from a sample.

22. A method for conducting a multiplex assay on a sample, the method involving:
providing dye-ligand conjugates wherein each ligand is bound to a different dye and is specific to an analyte,
forming a mixture by mixing the dye-ligand conjugates with the sample in order to allow the dye-ligand conjugates to bind with any specific analyte present in the sample and removing unbound dye-ligand conjugates;
measuring a spectrum of the mixture using a transduction technique;
identifying which analyte(s) is/are present in the sample by comparison of the spectrum to a reference spectrum for each dye, the reference spectrum obtained from a sample in which the dye is at a reference concentration using the transduction technique, wherein each dye is identifiable at better than 90% sensitivity and 90% specificity in the presence of any other dye of the set throughout a range of concentrations of each of the two dyes of 0.6 to 1.5 of the respective dye's reference concentration.

23. A method of enabling targets in a sample to be identified using Raman spectroscopy apparatus, the method comprising supplying a kit according to claim 5 and using calibration samples comprising the dyes at the reference concentrations to generate with the Raman spectroscopy apparatus a set of reference spectra to be used in identifying the dyes in the kit, and storing the reference spectra for later use when analyzing a Raman spectrum generated using the Raman spectroscopy apparatus when conducting a multiplex assay using the kit.

24. A method of selecting X dyes among N dyes comprising generating SERRS spectra for dye sets, each dye set comprising one or more dyes selected from the N dyes, and calculating a figure of merit indicative of a chance of identifying, from the SERRS spectra, correctly as present the one or more dyes in each set and/or incorrectly as present the dyes absent from each set and selecting the X dyes based upon the figure of merit.

25. A method according to claim 23, wherein the dye sets include dye sets comprising two or more dyes.

26. A method according to claim 25 comprising establishing a reference concentration for each dye, wherein the SERRS spectra generated for each dye set are based upon the dye set comprising one or more major dyes and a minor dye, wherein a ratio of a concentration of the or each major dye relative to the major dye's reference concentration is greater than a ratio of a concentration of the minor dye relative to the minor dye's reference concentration.

27. A method according to claim 26, wherein the figure of merit comprises simulating for each dye set, a limit of concentration of the minor dye at which the minor dye can be identified with a sensitivity greater than a set performance criteria when in the presence of one or more major dyes; and selecting the X dyes comprises selecting the dyes based upon the limit of concentration.

28. A method according to claim 26 wherein the figure of merit comprises, for each dye set, determining specificity and/or sensitivity at at least one concentration, and selecting the X dyes comprises selecting the dyes based upon the determined specificity and/or sensitivity.

29. A method according to claim 25, comprising selecting a subset of dyes based upon a figure of merit calculated for m-plex dye sets then selecting dyes from the subset based upon a figure of merit calculated for p-plex dye sets formed from combinations of the subset of dyes, wherein each m-plex dye set comprises fewer dyes than each p-plex dye set.

30. A method according to claim 29, wherein the m-plex dye sets are simplex dye sets and the p-plex dye sets are duplex dye sets.

31. A method according to claim 25, wherein the SERRS spectra are simulated from data indicative of the variability of SERRS spectra obtainable from each dye of the set.

32. A method according to claim 31, wherein the variability data comprises SERRS spectra for each dye obtained experimentally under different conditions.

33. A dye set selected using the method of claim 25.
